(19) United States
(12) Patent Application Publication
(10) Pub. No.: US 2023/0277371 A1
(43) Pub. Date: Sep. 7, 2023

(54) HEATING ELEMENT COMPOSITION, AND METHOD AND DEVICE OF MANUFACTURING EYE MASK PACK

(71) Applicant: HUMMINGAVIS CO.,LTD., Gyeongju-si, Gyeongsangbuk-do (KR)

(72) Inventors: Sung Ryong YU, Cheonan-si, Chungcheongnam-do (KR); Ju Taek KIM, Asan-si, Chungcheongnam-do (KR)

(21) Appl. No.: 18/008,262

(22) PCT Filed: Oct. 28, 2020
(86) PCT No.: PCT/KR2020/014786

§ 371 Date: Dec. 5, 2022

(30) Foreign Application Priority Data
Jun. 5, 2020 (KR) 10-2020-0068566
Aug. 13, 2020 (KR) 10-2020-0101517

(51) Int. Cl. A61F 7/02; C09K 5/18

(52) U.S. Cl. CPC A61F 7/0241; C09K 5/18; A61F 2007/038

(57) ABSTRACT

The present invention relates to a heating element composition, and a method of manufacturing an eye mask pack, which shows that a heating temperature is regular, and heat is uniformly generated, a heating pack is able to be worn in a state of coming into close contact even with a curved surface, a preservation period is long, and the heating temperature and time for which heat is generated can be adjusted minutely. Also, the present invention relates to a device of manufacturing an eye mask pack which is capable of manufacturing self-heating packs rapidly even without the use of nitrogen.

### TECHNICAL FIELD

The present invention relates to a heating element composition, and a method of manufacturing an eye mask pack, which show that a heating temperature is regular, heat is generated uniformly, the mask pack can be worn in a state of coming into close contact even with a curved surface, a storage period is long, and the heating temperature and time for which heat is generated can be adjusted minutely. Also, the present invention relates to a device of manufacturing an eye mask pack, which is capable of rapidly manufacturing self-heating packs even without the use of nitrogen.

### BACKGROUND ART

Dryness of eyes occurs at the time of concentrating people's attention on looking at a computer, and a mobile display for a long time, car driving, reading, and so on due to eye strain, and the evaporation of tears caused by the eye strain. In order to recover the eyes from this eye strain, a patch for warm fomentation attached to regions around the eyes after being heated using a microwave oven, a patch for cold fomentation applied to regions around the eyes after being preserved in a refrigerator, and so on have been introduced.

These patches are intended for providing the regions around the eyes with the warm fomentation and the cold fomentation, and are not effective at all to recover the eyes from the dryness of eyes and so on because they fails to restore moisture.

In particular, in case of the patch for warm fomentation, it is problematic in that since it is difficult to apply heat to the patch at an appropriate temperature, the patch may put a user in damage, like a burn and so on, and since the patch is heated in a state of being located in the center of the eyes, the cornea which is weak in heat can be burned, and a fixed temperature of the patch cannot be maintained continuously.

Also, in case of a self-heating material, in order to maintain a shape of the heating material, an auxiliary means, like facial tissues, a non-woven fabric, and so on, is used, but the heating material may enter a user's eyes when the heating material is separated from the auxiliary means due to an impact from the outside, or his or her carelessness

Thus, through Korean Patent Application No. 10-2013-0135881, the present applicant has ever disclosed a self-heating composition in which iron powder, carbon powder, aqua slush, polymer substance (polyvinyl alcohol), loess powder, pulp are mixed with distilled water.

The iron powder generates heat by reacting to atmospheric oxygen as shown in Reaction Formula (1) below:

2Fe+3O₂→Fe₂O₃+heat (1)

A heating patch may be manufactured using the self-heating composition, and a mask pack may be manufactured using this heating patch.

Accordingly, when a self-heating type mask pack is manufactured, it is important that the heating patch isn't exposed to the air.

If the heating patch is exposed to the air, it will start to generate heat by reacting to oxygen. Also, if a heating reaction has already occurred in a manufacturing process, it is problematic in that the heating patch would have failed to show its function due to lack of a heating reaction at the time of using of the user.

In order to solve such a problem, as shown in FIG. 1, an eye mask pack EMP manufactured by “a device and a method of manufacturing a self-heating mask pack” disclosed in Korean Patent Reg. No. 10-1539000 is configured in such a manner that a upper layer cover and a lower layer cover are stacked on an upper portion and a lower portion of a heating patch HP, respectively.

The upper layer cover shows that a non-breathable sheet USH1, an inside sheet ISH, and an earing sheet ESH are successively piled up from the upper portion of the heating patch PH. Holes are formed in the earing sheet ESH so that a user's ears can get caught in the earing sheet ESH.

Also, the lower layer cover shows that another non-breathable sheet USH2 furnished at the lower portion of heating patch PH, and an outside sheet and are successively stacked.

Accordingly, the heating patch HP is sealed by the non-breathable sheets USH1 and USH2 of the upper layer cover and the lower layer cover not to be exposed to the air.

Meanwhile, the conventional manufacturing method of the aforesaid mask pack comprises: step 1) of supplying a heating patch; step 2) of adhering a non-breathable sheet to a lower portion of the heating patch; step 3) of spraying a saline solution onto the heating patch at a nitrogen atmosphere; step 4) of sealing the heating patch by adhering another non-breathable sheet to an upper portion of the heating patch; step 5) of adhering an outside sheet and an inside sheet to the heating patch sealed by the non-breathable sheets; step 6) of manufacturing the eye mask pack by cutting the inside sheet and the outside sheet in a fixed shape; and step 7) of inserting the mask pack into a pouch, and sealing it.

As shown in FIG. 2, with respect to the mask pack manufactured by the method described above, since the heating patch is dried after the saline solution is sprayed upon the heating patch, the heating patch has fixed strength in a flat state, and is cured.

On the contrary, a face from the human body is not flat, and has a three-dimensional shape with bending. Accordingly, when a user puts the mask pack on his or her face in a state of his or her ears being caught in the earing sheet ESH, it is problematic in that the heating patch HP which has strength and is cured fails to come into close contact with regions around his or her eyes.

Also, it has been disadvantageous in that frequency in occurrence of a negligent accident due to the use of nitrogen is high, and since a chamber filled with nitrogen opens often, it has been problematic in that the corresponding nitrogen should be filled continuously, and a problem of maintenance and repairing also occurs due to loss and so on.

### DETAILED DESCRIPTION OF THE INVENTION

### Technical Problem

The present invention has been devised for solving the aforesaid problems, and an object of the present invention is to provide a heating element composition, and a method of manufacturing an eye mask pack using the heating element composition, which show that since a composition ratio is regularly maintained, a heating temperature is regular, and heat is generated uniformly.

Another object of the present invention is to provide a heating element composition, and a method of manufacturing an eye mask pack using the heating element composition, which show that since a the heating pack has strength and is formed in a powder form rather than a stick form, the heating pack can be worn in a state of coming into close contact even with a curved surface.

Another object of the present invention is to provide a heating element composition and a method of manufacturing an eye mask pack using the heating element composition, which show that since a heating pack is in a dry state, the possibility of oxidation is small, and a preservation period is long.

Another object of the present invention is to provide a heating element composition and a method of manufacturing an eye mask pack using the heating element composition, which show that since a heating pack is formed in a powder form, it can be worn in a state of coming into close contact with a three-dimensional region from the human body, like a face and the like.

Another object of the present invention is to provide a heating element composition and a method of manufacturing an eye mask pack using the heating element composition, which show that since a mixing ratio of the composition is precisely adjusted, a heating temperature and time for which heat is generated can be adjusted minutely.

The other object of the present invention is to provide a manufacturing device, which enables quality of mask packs as goods to be secured in such a manner as to carry out forming for the self-heating mask packs rapidly even without the use of a chamber filled with nitrogen, and then to package them.

### Solution for Solving the Problem

In order to solve the aforesaid technical problems, it is preferable that a heating element composition according to the present invention comprises: 50 to 100 parts by weight of carbon powder; 1 to 50 parts by weight of loess powder; 1 to 50 parts by weights of pulp powder; and 5 to 50 parts by weight of salt with respect to 100 parts by weight of a first piece of iron powder. Also, it is preferable that the heating element composition further comprises 1 to 50 parts by weight of a high adsorptive resin with respect to 100 parts by weight of the first piece of iron powder. Also, it is preferable that the heating element composition further comprises 200 to 500 parts by weight of a second piece of iron powder with respect to 100 parts by weight of the first piece of iron powder.

Meanwhile, a method of manufacturing an eye mask pack in which a heating element composition is contained according to the present invention, comprising: step 1) of making a first mixture by mixing carbon powder in a first piece of iron powder; step 2) of making a second mixture by mixing loess powder, pulp powder, and a high adsorptive resin in the first mixture; step 3) of making a heating pack by sealing the second mixture; step 4) of preserving the heating pack sealed in a vacuum pack in a state of being wound on a reel; and step 5) of manufacturing the mask pack by releasing a sealing state of the heating pack, and putting salt and water.

Also, a device of manufacturing an eye mask pack according to the present invention comprises: a pack formation part configured to carry out forming for a heating pack in which a heating part is furnished; a brine provision part configured to supply brine to the heating part of the heating pack; a first adhesion part configured to carry out forming for a first forming sheet by thermally adhering a breathable sheet and a non-breathable sheet to an upper portion and a lower portion of the heating pack, respectively; a second adhesion part configured to carry out forming for a second forming sheet by thermally adhering an outside sheet and an inside sheet to a top surface and a bottom surface of the first forming sheet, respectively; a third adhesion part configured to adhere an earring sheet, which is perforated, to the top surface of the second forming sheet through ultrasonic; and an outward appearance formation part configured to carry out forming for a mask pack in such a manner as to perforate the second forming sheet.

### Effect of the Invention

According to the present invention, it is effective in that a composition ratio is regularly maintained so that a heating temperature is regular, and heat is uniformly generated.

In particular, a heating pack has strength, and is formed in a powder form rather than a stick form so as to be worn in state of coming into close contact even with a curved surface.

Also, since the heating pack is in a dry state, the possibility of oxidation is small, so a preservation period is long.

Also, a mixing ratio of the composition is precisely adjusted so that a heating temperature and time for which heat is generated can minutely be adjusted.

Also, since the heating pack is the powder form, it is effective in that the heating pack can be worn in a state of coming into close contact with a three-dimensional region from the human body, like a face and the like.

Also, after forming for self-heating mask packs is carried out rapidly even without the use of nitrogen, the mask pack can be packaged, so a negligent accident can be prevented from occurring, and convenience for maintenance and repairing can also be provided.

Also, it is effective in that in order to control the position of an outside sheet, supply of other sheets is collectively controlled according to supply of the outside sheet.

### BRIEF DESCRIPTION OF THE DRAWINGS

FIG. 1 represents a conventional eye mask pack.

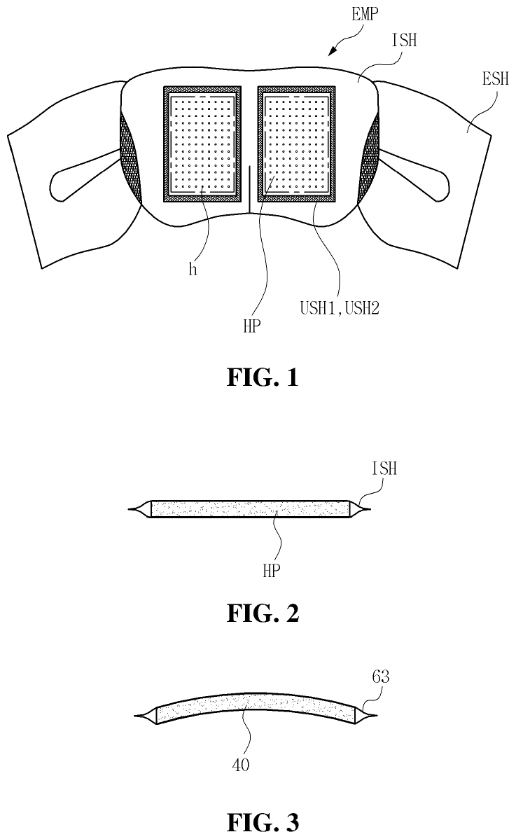

FIG. 2 is a cross-sectional view showing the eye mask pack shown in FIG. 1.

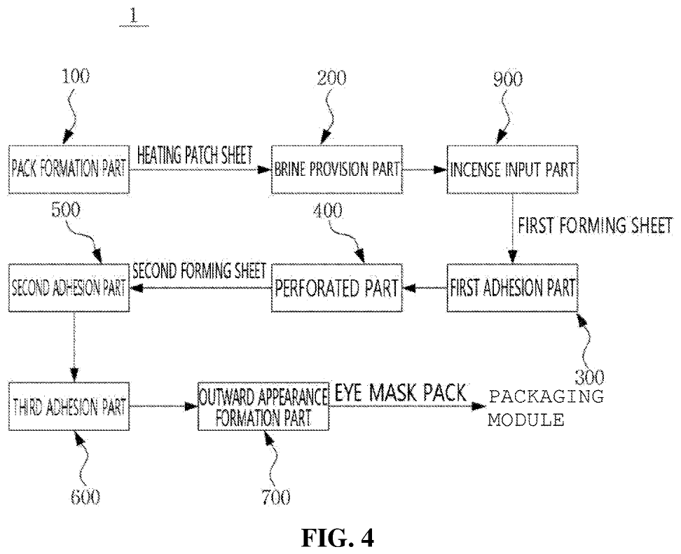

FIG. 3 is a cross-sectional view showing an eye mask pack according to the present invention.

FIG. 4 is a conceptual view concerning a device of manufacturing a self-heating eye mask pack according the present invention.

FIG. 5 is a view showing a patch formation part with respect to the manufacturing device of the self-heating eye mask pack according to the present invention.

FIG. 6 is a view showing a mixer concerning FIG. 5.

FIG. 7 is a view showing a powder provision member concerning FIG. 5.

FIG. 8 is an operational state view concerning a spreading means concerning FIG. 7.

FIG. 9 is a view showing a cutting member concerning FIG. 6.

FIG. 11 is a view showing a brine provision part and a first adhesion part with respect to the manufacturing device of the self-heating eye mask pack according to the present invention.

FIG. 11 is a view showing a perforated part and a second adhesion part with respect to the manufacturing device of the self-heating eye mask pack according to the present invention.

FIG. 12 is a view showing a third adhesion part and an outward appearance formation part with respect to the manufacturing device of the self-heating eye mask pack according to the present invention.

FIG. 13 is a view illustrating the self-heating mask pack with respect to the manufacturing device of the self-heating eye mask pack according to the present invention.

FIG. 14 to FIG. 16 are views showing a position control sensor part with respect to the manufacturing device of the self-heating eye mask pack according to the present invention.

### MODE FOR CARRYING OUT THE INVENTION

Hereinafter, the constitutions and operations of the exemplary embodiments according to the present invention are described in detail.

A heating element composition according to the present invention comprises: a first piece of iron powder; a second piece of iron powder; carbon powder; loess powder; pulp powder; a high adsorptive resin; and salt.

Such a heating element composition is dried so that a heating pack can be manufactured, wherein the heating pack is manufactured so as to be closely attached onto a bendy region, like a face and so on, because the composition is accommodated in a powder form, and sealing is carried out so that a mask or an eye mask can be manufactured.

The first piece of iron powder and the second piece of iron powder generate heat by reacting on atmospheric oxygen as shown in Reaction Formula (1) below:

2Fe+3O₂→Fe₂O₃+heat (1)

In general, in case of the first piece of iron powder used in the heating element, although powder composed of 100 meshes or below is used, a heating reaction may occur rapidly, and a corneal burn may be caused by heating which reaches 70° C. or more. Thus, the present invention is characteristic in that the first piece of iron powder and the second piece of iron powder which are different from each other with respect to particle sizes thereof are included so that the iron powder maintains a temperature of 40 to 50° C.

That is, in the present invention, the first piece of iron powder corresponding to 200 to 500 meshes, and the first piece of iron powder corresponding to 50 to 180 meshes are included. Although the first piece of iron powder is mainly in charge of heating from the heating element composition, if only the first piece of iron powder is included without the second piece of iron powder, it will be problematic in that heat is generated for a short time at a high temperature. Accordingly, in order to cause heat to be generated for a long time at a temperature of 40 to 50° C. while maintaining an appropriate total amount of heat generated, the second piece of iron powder is included.

In the present exemplary embodiment, the first piece of iron powder is composed of 325 meshes, and the second piece of iron powder is composed of 100 meshes.

The carbon powder functions as a catalyst for activating a chemical reaction like said Reaction Formula (1). It is preferable that in the heating element composition, the carbon powder is contained in an amount of 50 to 100 parts by weight with respect to 100 parts by weight of the first piece of iron powder. When the carbon powder is contained in the amount of less than 50 parts by weight with respect to 100 parts by weight of the iron powder, a speed of the chemical reaction of Reaction Formula (1) gets slow, and the amount of generated heat is also small. On the contrary, when the carbon powder is contained in the amount of exceeding 100 parts by weight with respect to 100 parts by weight of the iron powder, it is problematic in that no chemical reaction of Reaction Formula (1) occurs, or a reaction speed gets slow.

In the present invention, the carbon powder means activated carbon, and although the activated carbon is largely divided into plant-based activated carbon, and coal-based activated carbon, in the present invention, it is preferable to use the plant-based activated carbon.

Since the loess powder has a porous structure, and comprises calcium carbonate (lime) among the ingredients of loess, when it is mixed with an aqueous solution, stickiness occurs, and due to this, the shape of a heating material can be maintained. The loess powder is mixed in an amount of 1 to 50 parts by weight with respect to 100 parts by weight of the first piece of iron powder, and when the loess powder is mixed in the amount of less than 1 part by weight, or in the amount of exceeding 50 parts by weight, a deflection in heating temperatures occurs largely.

The pulp powder is divided into wood pulp and non-wood pulp, and in the present invention, the wood pulp is used, so it is preferable to use pulp resulting from pulverizing the pulp in a general paperboard form so that its form is converted into a fluff form.

In case of the pulp in the fluff form, although it is not in direct relation to a heating reaction, in addition to loess, a fibrous material of the pulp is used in maintaining the shape of the heating component.

Also, the pulp powder may be cellulose.

It is preferable to contain 1 to 50 parts by weight of the pulp with respect to 100 parts by weight of the iron powder, and when the amount of the pulp exceeds 50 parts by weight, although it is favorable to maintain the shape of the heating element, a heating speed and a heating temperature are not appropriate, and when the amount of the pulp is less than 1 part by weight, it is difficult to maintain the shape of the heating element because the shape of the heating element gets scattered.

The present invention comprises a high absorptive resin, and in the present exemplary embodiment, the high absorptive resin is an acrylic acid polymer, or sodium salt.

Since the high adsorptive resin is included, the generation of heat occurs in its early stage rapidly, and after the generation of heat is completed, the shape of the heating element is maintained without being broken easily.

The high adsorptive resin is mixed in an amount of 1 to 50 parts by weight with respect to 100 parts by weight of the first piece of iron powder, and in case that the amount exceeds 50 parts by weight, an initial heating speed may get fast excessively, or the generation of heat may decrease, and in case that the amount is less than 1 part by weight, it is difficult to expect that the effect as described above will occur.

The salt is intended to generate warm heat steam using heat generated when the first piece of iron powder and the second piece of iron powder are oxidized according to said Reaction Formula (1) above. At this time, the warm heat steam generated is supplied to eyes and regions around the eyes so that the eyes can be moisturized, and warm fomentation can be performed. It is preferable that in the heating element composition, the salt is contained in an amount of 1 to 20 parts by weight with respect to 100 parts by weight of the iron powder, and when the amount of the salt exceeds 20 parts by weight, time for which heat is generated based on Reaction Formula (1) is delayed, or a heating temperature gets low. On the contrary, when the amount of the salt is less than 1 part by weight, it is problematic in that time for which heat is generated is shortened.

Hereinafter, a method of manufacturing a heating pack, and a method of manufacturing an eye mask pack using the heating pack are described.

First, the manufacturing method of the heating pack comprises making a first mixture by mixing carbon powder in a first piece of iron powder, and making a second mixture by mixing loess powder and pulp powder in the first mixture.

As described above, after the second mixture is made, it is sealed, so the heating pack is manufactured.

Specifically explaining the manufacturing method of the heating pack, first the second mixture is uniformly applied onto a pulp fabric.

Next, the second mixture applied onto the pulp fabric is covered with a non-woven fabric.

Last, the non-woven fabric located at the upper portion, and the pulp fabric located at the lower portion adhere to each other in a state of putting the second mixture therebetween, and in the present exemplary embodiment, thermal adhesion is carried out.

However, unlike this, with respect to a publicly-known means, a pulp fabric located at the upper portion, and a not-woven fabric located at the lower portion adhere to each other, so a heating pack can be manufactured.

The heating pack manufactured as described above is preserved in a vacuum pack in a state of being wound in a reel.

Next, a sealing state of the heating pack preserved in the vacuum pack is released, and salt and water are put in the pack, so a mask is manufactured.

It is preferable to put 50 to 200 parts by weight of the water with respect to 100 parts by weight of the first piece of iron powder.

According to the present invention, as described above, no water is contained in the heating pack, but compositions are accommodated in a powder form. Accordingly, unlike the conventional art, the heating pack is not in a state of being cured in a fixed form.

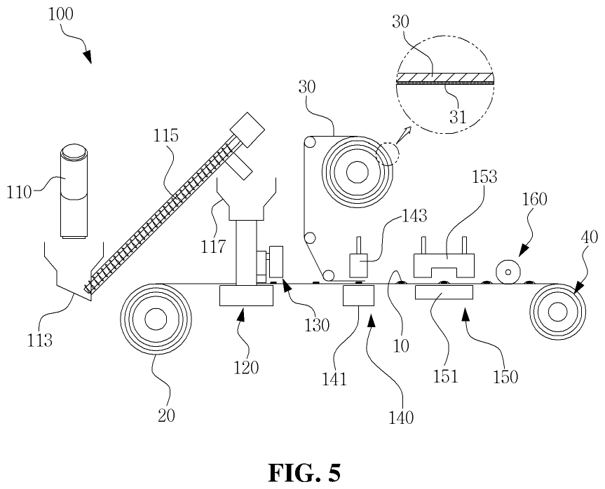

Accordingly, as shown in FIG. 3, unlike the conventional art, since the heating pack is formed in the powder form, it can be worn on three-dimensional regions from the human body, like a face and so on, in a state of coming into close contact therewith.

Hereinafter, a device of manufacturing an eye mask pack according to the present invention (hereinafter referred to as “the manufacturing device” briefly) is described in detail with reference to the accompanying drawings.

Prior to the description, it should be kept in mind that in the manufacturing device 1 according to the present invention, a packaging part configured to package an eye mask pack 80, forming for which is completed, through a vacuum may be installed, and the packaging part uses quotations from the contents concerning a packaging module 90 suggested in Korean Patent Reg. No. 10-1539000 entitled “a device of manufacturing a self-heating mask pack” invented by the present applicant.

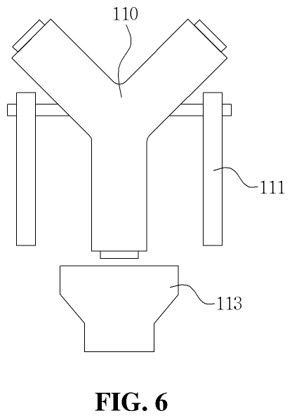

As shown in FIG. 4, the manufacturing device 1 according to the present invention largely comprises: a pack formation part 100; a brine provision part 200; a first adhesion part 300; a perforated part 400; a second adhesion part 500; a third adhesion part 600; an outward appearance formation part 700; and an incense input part 900.

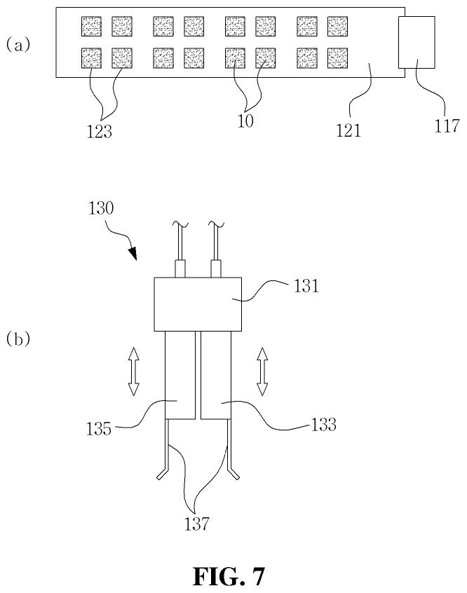

More specifically explaining, the pack formation part 100, as illustrated in FIG. 5, is a constitution intended for supplying the heating pack 40 in a roller (or a reel) form, which can be unwound, to the brine provision part 200 which will be described later, after carrying out forming for the heating pack 40, and the pack formation part comprises: a mixer 110; a powder provision member 120; a thermal adhesion member 140; a punched member 150; and a cutting member 160.

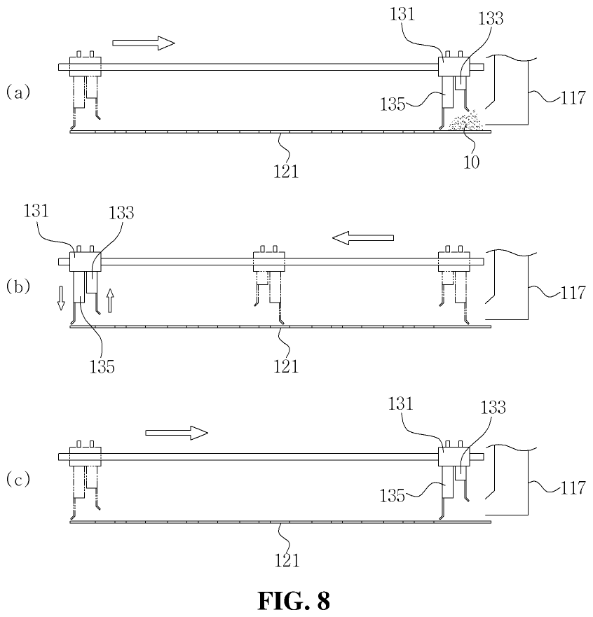

For example, referring to FIG. 6 more, the mixer 110 is a constitution intended for producing a heating part 10 by mixing a large number of powder compositions consisting of iron powder, carbon powder, aqua slush, a polymer material (polyvinyl alcohol), loess powder, pulp, and so on with one another.

For this, the mixer 110 is entirely formed in an alphabet “Y”-like shape, and a door which can open and shut is formed in each of an upper portion and a lower portion of the mixer so that a lot of powder can be put in the inside via the door located at the upper portion, and the heating part 10 formed by mixing can be discharged in a powder form via the door located at the lower portion.

Both sides of the mixer 110 are combined with a support body 111 to be rotatable through a rotary axis, and repeatedly rotate in an up-and-down direction centering around the rotary axis by driving of a driving motor so that the large number of power compositions put in the inside of the mixer can easily be mixed.

At this time, it is preferable that the mixer 110 is axially combined with the support body 111 so that load of the lower portion can get heavier in order to enable a return to an alphabet “Y”-like figure.

By rotation of the mixer 110, the heating part 10 in the powder form is discharged through the door furnished in the lower portion of the mixer 110, and the heating part 10 discharged has a structure in which the heating part is supplied to a first hopper 113, and is then supplied in each fixed amount to a second hopper 117 by the rotation of a supply screw 115 having an angle of inclination.

A lower portion of the second hopper 117 which is open is disposed to be adjacent to one side of a pattern tray 121 so that the heating part 10 supplied to the inside of the second hopper 117 can be discharged.

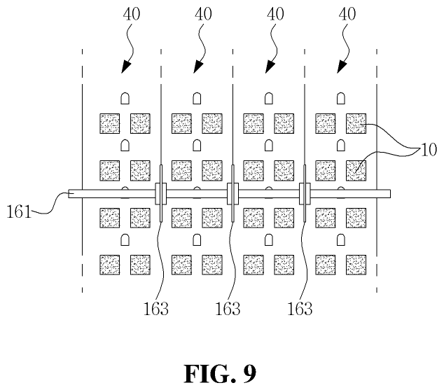

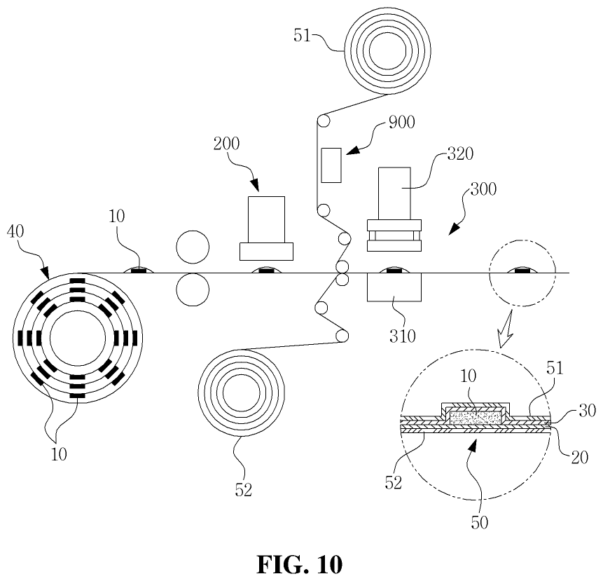

The powder provision member 120, as illustrated in FIG. 7 and FIG. 8, is a constitution intended for causing the heating part 10 to make fixed patterns on a top surface of the pulp fabric 20 by spreading the heating part 10 in the powder form supplied via the open lower portion of the second hopper 117 onto the pattern tray 121 uniformly.

The pattern tray 121 is formed to have a plate-like shape, wherein input holes 123 through which the heating part 10 passes in a lengthwise direction are formed, and the heating part 10 transferred to a lower portion of the pattern tray 121 via the input holes 123 can stably be attached onto the top surface of the pulp fabric 20 so as to have uniform patterns.

To do so, the pattern tray 121 is formed in such a manner that one side of the pattern tray has a wider area than another side thereof, so receiving of the heating part 10 can be realized.

Moreover, the powder provision member 120 is able to perform a sliding, reciprocating movement so that the heating part discharged to one side of the pattern tray 121 via the second hopper 117 can uniformly be supplied via the large number of input holes 123, and a spreading means 130 which is movable up and down is installed, wherein the spreading means 130 comprises: a moving body 131; a first cylinder 133; a second cylinder 135; and a distribution plate 137.

The moving body 131 is installed so as to perform a reciprocating movement along a bracket or a rail disposed parallel to an upper portion of the pattern tray 121 via a means for movement, like a motor, a cylinder, and so on, and the first cylinder 133 and the second cylinder 135 are installed at a lower portion of the pattern tray so as to move upward and downward individually according to air pressure or oil pressure supplied from the outside.

The distribution plate 137 is installed at a lower portion of the first cylinder 133 and the second cylinder 135 so as to have a plate-like shape, wherein a bending part having a fixed angle of inclination in a direction which is opposite to another direction intended for spreading the heating part 10 onto the pattern tray 121 is formed.

As shown in FIG. 8, the spreading means 130 is first on standby from one side (the left side with the drawing as a standard) of the pattern tray 121, and when the heating part 10 is supplied via the second hopper 117 which is open from another side (the right side with the drawing as a standard) which is opposite to one side, the moving body 131 moves to slide in a direction of another side of the pattern tray 121.

At this time, as the first cylinder 133 furnished in a lower portion of the moving body 131 moves in the direction of another side of the pattern tray 121 in a state of going up, a pile of the heating part 10, which is piled up on the distribution plate 137 protruding from a lower portion of the second cylinder 135 into a downward direction, is prevented from scattering.

Then, the first cylinder 133 which moved to another side of the pattern tray 121 moves downward again, and in a state of the second cylinder 135 going up, the moving body 131 moves to slide to one side which is its first position.

In this process, the distribution plate 137 formed at a lower portion of the first cylinder 133 causes the heating part 10 to be distributed onto the large number of input holes 123 formed in the pattern tray 121 while sweeping the pile of the heating part so that the heating part 10 can form uniform patterns on the top surface of the pulp fabric 20.

Meanwhile, in the process of distributing the heating part 10 to the pulp fabric 20 via the distribution plate 137 of the first cylinder 133, since the second cylinder 135 is in a state of going up, it doesn't intervene in the distribution of the heating part 10.

Also, after the distribution of the heating part 10 is primarily completed, the second cylinder 135 moves downward, and the moving body 131 moves in the direction of another side of the pattern tray 121 once again in a state of the first cylinder 133 going up, so the residue of the heating part 10, which fails to be distributed, can be put into the input holes 123,

The thermal adhesion member 140, as illustrated in FIG. 5, is a constitution intended for thermally adhering a non-woven fabric 30 to the top surface of the pulp fabric 20 on which the heating part 10 is furnished, and comprises a lower plate 141 and an upper plate 143.

The pulp fabric 20 is disposed at an upper portion of the lower plate 141 so as to be transferred. Also, the non-woven fabric 30 thermally adheres to the top surface of the pulp fabric 20 as the upper plate 142 disposed at an upper side of the lower plate 141 moves downward. Accordingly, in order for the heating part 10 in the powder form furnished on the pulp fabric 20 to be prevented from being separated from the pulp fabric, adhesion for an edge part of the heating part is performed, and forming for the heating pack 40 to be supplied to the brine provision part 200 which will be described later is performed.

At this time, it is preferable to realize the facility of thermal adhesion by treating an outer surface of the non-woven fabric 30, namely a surface facing the pulp fabric 20, with polyethylene (PE) coating 31.

In the exemplary embodiment of the present invention, a polyethylene (PE) or polyethylene terephthalate (PET) material may be applied into the non-woven fabric 30. At this time, since PE has a low melting point, it is not necessary to treat the non-woven fabric with PE coating 31 separately, but wrinkles may occur in a process of carrying out thermal adhesion through the thermal adhesion member 140.

On the contrary, in case of PET, since it is high in melting point, thermal adhesion is not carried out smoothly, so it is preferable to treat the surface of the PET non-woven fabric 30 facing the pulp fabric 30 with PE coating 31, and it is advantageous in that no wrinkles occur because an influence by heat is small.

The punched member 150 is a constitution intended for making perforations which enable the heating pack 40 to be caught in the ridge part of a wearer's nose, and comprises a lower punched plate 151 and an upper punched plate 153.

As illustrated, the lower punched plate 151 is disposed so that the heating pack 40 is transferred to an upper portion of the lower punched plate, and the upper punched plate 153 is installed in a structure which enables the upper punched plate to move upward and downward from an upper side of the lower punched plate 151, thereby perforating the heating pack 40 through upward and downward movements.

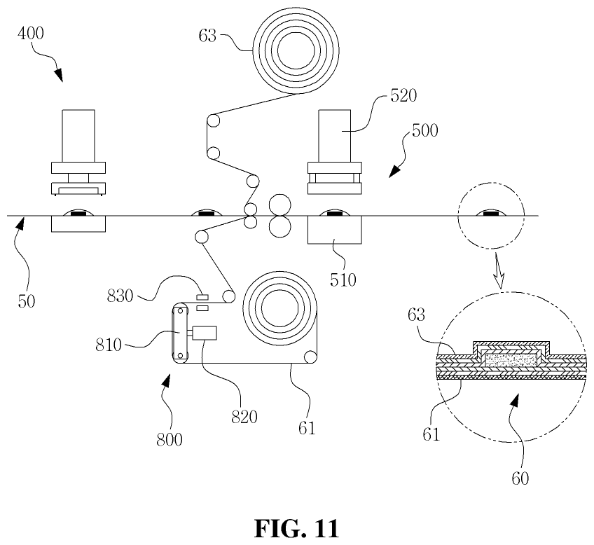

The cutting member 160, as illustrated in FIG. 5 and FIG. 9, is a constitution intended for cutting the perforated heating pack 40 in a fixed width, and comprises a rotary table 161 and blades 163.

The rotary table 161 entirely composed in a pillar-like shape having a fixed length is installed in such a manner that any one side of the rotary table can rotate thanks to a driving motor, and the large number of blades 163 are formed to have fixed intervals so that the plurality of heating packs 40 can be manufactured through cutting planes of the blades 163.

Moreover, although it is not illustrated, the heating packs which are cut by the blades, respectively, are installed so as to be wound on a rotatable roller, and thus the heating packs 40 are individually installed in the brine provision part 200 which will be described later 200 so as to be supplied.

Furthermore, the brine provision part 200, as illustrated in FIG. 10, is a constitution intended for providing the heating parts 10 of the heating packs 40 obtained by the aforesaid pack formation part 100 with brine, and has a pump configured to supply the brine, a nozzle configured to spray the brine, and a valve configured to adjust a spraying amount by being mounted to the nozzle. The brine contains salt and water, and as one example, a saline solution may be used.

Meanwhile, according to some exemplary embodiments, discriminately with the conventional arts, since the inside of the brine provision part 200 is prepared in a nitrogen atmosphere, after the brine is provided, the brine provision part conducts operation in a chamber so that the heating part 10 doesn't cause a heat reaction by coming into contact with the air outside. However, according to some exemplary embodiments, since the manufacturing device 1 may manufacture mask packs within several minutes required for reaching a process of packaging using pouches through a packaging module (not drawn) from the brine provision part 200, it is advantageous in that the production process may be carried out without a trouble in a state of the corresponding chamber being omitted.

Of course, it would have been obvious that before the heating packs manufactured so as to be unwound from the roller via the pack formation part 100 are supplied to the brine provision part 200, the heating packs are packaged separately so as to blocked from coming into contact with the air outside, or are preserved in a storing place suitable for such a condition separately.

Furthermore, the first adhesion part 300 is a constitution for adhering a breathable sheet 51 and a non-breathable sheet 52 to an upper portion and a lower portion of the heating pack 40 to which brine is supplied, respectively, and comprises an upper worktable 320 and a lower worktable 310.

At this time, an odoriferous solution is applied onto an outer surface of the breathable sheet 51 via the incense input part 900, and for example, it is preferable that the incense input part 900 applies the odoriferous solution in a liquid state onto the outer surface of the breathable sheet 51 via an application means (not drawn), like a brush, a roller, or the like, which comes into contact with the outer surface of the breathable sheet 51 so as to adhere to the non-breathable sheet 52 through the first adhesion part 300. However, the odoriferous solution may be applied onto the non-breathable sheet 52, and to do so, the incense input part 900 may be installed in an input path of the non-breathable sheet 52.

The upper worktable 320 is installed at an upper side of the lower worktable 310 so as to move upward and downward, and the heating pack 40 is transferred into between said two constitutions, wherein the non-breathable sheet 52 from the lower portion of the heating pack 40, and the breathable sheet 51 from the upper portion thereof are transferred together, so forming for the first forming sheet 50 can be carried out by thermal adhesion occurring accordingly as the upper worktable 320 moves downward.

Moreover, after the first forming sheet 50, forming for which is completed, is cut out by the perforated part 400, the first forming sheet may be furnished between the outside sheet 61 and the inside sheet 63 which thermally adhere to each other via the second adhesion part 500.

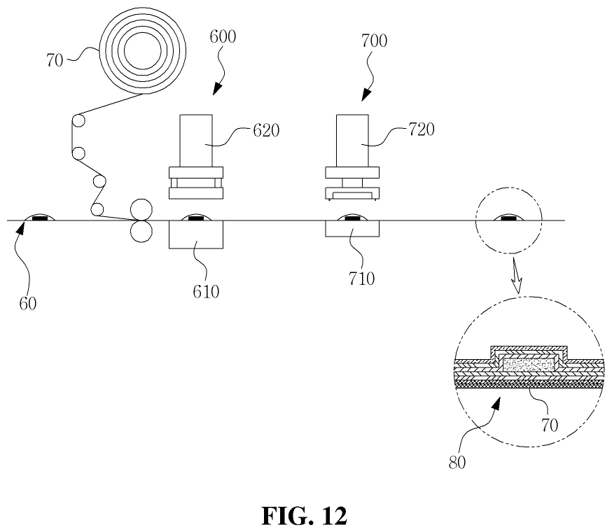

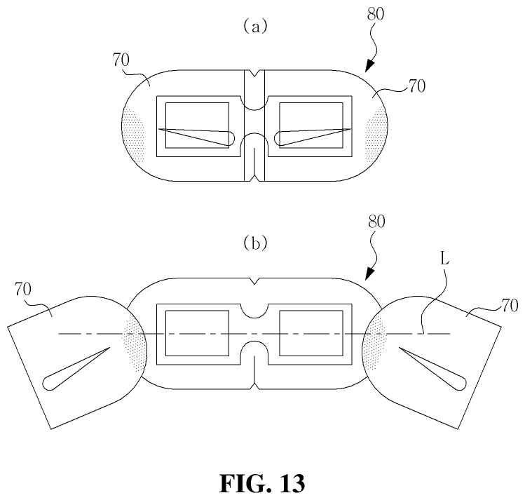

As shown in FIG. 11, the second adhesion part 500 is a constitution intended for carrying out forming for the second forming sheet 60 composed in an eyepatch-like shape, in which the earing sheet 70 is omitted, by thermally adhering the outside sheet 61 and the inside sheet 63 to the top surface and the bottom surface of the first forming sheet 50 which are punched by the perforated part 400, and the second adhesion part comprises a lower forming table 510 and an upper forming table 520.

For example, the upper forming table 520 is guided from an upper side of the lower forming table 510 so as to move upward and downward, and the first forming sheet 50 is disposed between said two constitutions so as to be transferred. Also, the outside sheet 61 which is unwound is disposed on the top surface of the first forming sheet 50, and the inside sheet 63 is disposed at the lower portion so that forming for the second forming sheet 60 can be carried out by thermal adhesion of the upper forming table 520 which moves downward.

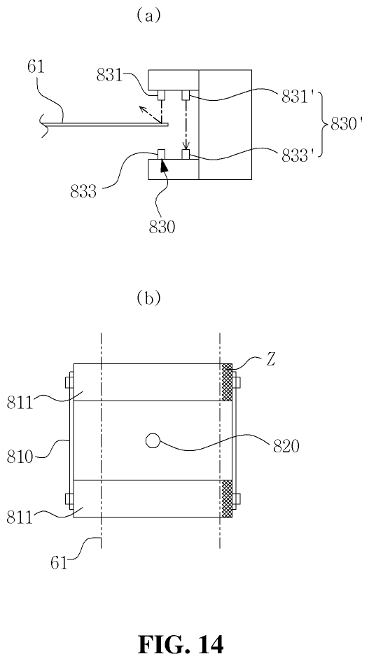

Furthermore, the third adhesion part 600, as illustrated in FIG. 12, is a constitution intended for adhering the earing sheet 70 which can be worn in a state of getting caught on a wearer's ears to an outer surface (preferably, a surface on which an inside sheet is furnished) of the second forming sheet 60, forming for which is completed, through ultrasound, and the third adhesion part 600 comprises a lower adhesion plate 610 and an upper adhesion plate 620.

To do so, the lower adhesion plate 610, and the upper adhesion plate 620 located at an upper portion thereof are installed so as to move upward and downward, and the second forming sheet 60 is installed between said two constitutions so as to be transferred. Also, the earing sheet 70 which can be unwound so as to come into surface contact with the inside sheet 63 of the second forming sheet 60 is supplied, so the structure which shows that the earing sheet adheres through ultrasound adhesion of the upper adhesion plate 620 is realized.

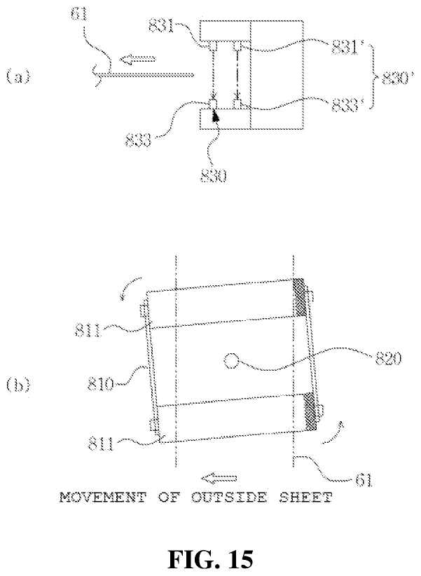

At this time, after the earing sheet 70, as illustrated in FIG. 13, unfolds with an optional, imaginary line L of the second forming sheet 60 as a standard, it is preferable that the earing sheet adheres to the second forming sheet so as to unfold to incline in a downward direction of the corresponding imaginary line L so that wearing can be facilitated through the holes formed in the earing sheet 70.

Referring to FIG. 12 again, the outward appearance formation part 700 is a constitution intended for carrying out forming for the eye mask pack 80 commercialized by perforating and cutting out the second forming sheet 60 to which the earing sheet 70 adheres, and the outward appearance formation part comprises a lower formation plate 710 and an upper formation plate 720.

For example, the lower formation plate 710, and the upper formation plate 720 located at the upper portion thereof are installed so as to move upward and downward, and perforate the outer surface of the second forming sheet 60, to which the earing sheet 70 before unfolding between said two constitutions adheres, through heat and so on, so forming for the eye mask pack 80 is finally carried out.

Meanwhile, referring to FIG. 11, in the present invention, except the pulp fabric 20 and the non-woven fabric 30, the outside sheet 61 which is unwound from the roller is controlled through the control signal of a control part so that supply of the inside sheets 63 which are unwound in a roller form to be completely identical to each other with a supply position of the outside sheets 61 as a standard, and supply of the earing sheet 70 can be decided.

To do so, the manufacturing device 1 which is the present invention, may further furnish a bracket with the position control sensor part 800 configured to sense the supply position of the outside sheets, and the position control sensor part 800 comprises a rotary bracket 810, a rotary motor 820, and a pair of sensing sensors 830.

For example, the outside sheet 61 is transferred to the second adhesion part 500 through a tension maintenance roller 811 which is axially combined with an upper portion and a lower portion of the rotary bracket 810 to be rotatable, and in this process, the pair of sensing sensors 830 in which a transmitting sensor 831 and a receiving sensor 833 are composed in one module in directions which face each other are installed at one side of the outside sheet 61 so as to sense the outside sheet 61.

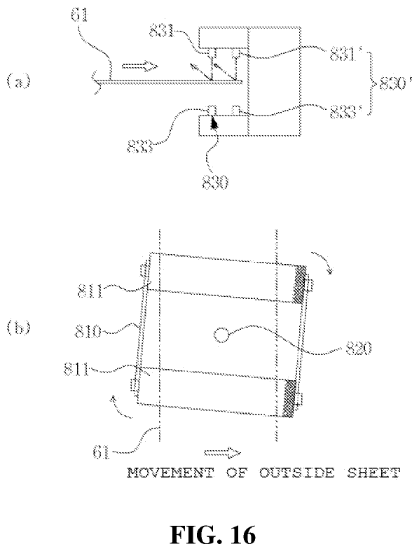

As one example, as shown in FIG. 14 to FIG. 16, the outside sheet 61 is supplied in a stable state of being uniform in a speed and a position thereof so as to be sensed by the first sensing sensor 830 in such a manner as to adjust one end of the outside sheet 61 with a reference district Z indicated at one side of the tension maintenance roller 811 as a standard, and the control part controls the supply of other sheets with this stable state as a standard.

In this process, when the outside sheet 61 deviates from sensing through the first sensing sensor 830 due to an external influence (e.g., mechanical vibration and so on) or is in a unstable state of being sensed by the second sensing sensor 830′, according to sensing information caused by the first sensing sensor 830 or the second sensing sensor 830′, the control part causes one end of the outside sheet 61 to reach the reference district by a rotation of the rotary bracket 810 in the normal direction (clockwise), the reverse direction, or the like through the rotary motor 820, thereby causing the outside sheet to move so as to reach a stable state, so uniform positions can be maintained with respect to all the sheets.

Of course, in the present invention, although it is described that the position control sensor part 800 is applied to the outside sheet 61 so that the positions to which the other sheets are supplied can uniformly be maintained, this is only one exemplary embodiment, and the position control sensor part can also be applied to the other sheets as needed, and can composed and installed of a plural number.

The manufacturing device 1 according to the present invention consisting of the constitutions as described above may package the self-heating eye mask pack 80 after carrying out forming for it rapidly even without the use of nitrogen discriminately with conventional devices, so an accident which compromises safety can be prevented from occurring, and convenience for maintenance and repairing can be provided.

Also, discriminately from conventional devices, since a position of the outside sheet 61 is controlled, it can be effect to collectively control the supply of other sheets to be matched with the supply position of the outside sheet.

### INDUSTRIAL APPLICABILITY

The present invention relates to an eye mask pack used in such a manner as to attach a heating pack by which self-heating is realized onto a region around the eyes, and is used in the industrial field to which a heating element composition for the eye mask pack, and a method and a device of manufacturing the eye mask pack pertain.

What is claimed is:

1. A heating element composition, comprising:
50 to 100 parts by weight of carbon powder,
1 to 50 parts by weight of loess powder,
1 to 50 parts by weights of pulp powder, and
5 to 50 parts by weight of salt with respect to 100 parts by weight of a first piece of iron powder.

2. The heating element composition of claim 1, further comprising 1 to 50 parts by weight of a high adsorptive resin with respect to 100 parts by weight of the first piece of iron powder.

3. The heating element composition of claim 1, further comprising 200 to 500 parts by weight of a second piece of iron powder with respect to 100 parts by weight of the first piece of iron powder.

4. A method of manufacturing a mask pack, with respect to the manufacturing method of the mask pack in which a heating element composition is contained, comprising:
step 1) of making a first mixture by mixing carbon powder in a first piece of iron powder;
step 2) of making a second mixture by mixing loess powder, pulp powder, and a high adsorptive resin in the first mixture;
step 3) of making a heating pack by sealing the second mixture;
step 4) of preserving the heating pack sealed in a vacuum pack in a state of being wound on a reel; and
step 5) of manufacturing the mask pack by releasing a sealing state of the heating pack, and putting salt and water.

5. The method of claim 4, wherein said step 3) comprises:
step 3-1) of applying the second mixture onto a pulp fabric;
step 3-2) of covering an upper portion of the second mixture with a non-woven fabric; and
step 3-3) of adhering the pulp fabric and the non-woven fabric to each other in a state of putting the second mixture therebetween.

6. The method of claim 5, wherein in said step 5), the water is put in an amount of 50 to 200 parts by weight with respect to 100 parts by weight of the first piece of iron powder.

7. A device of manufacturing an eye mask pack, comprising:
a pack formation part 100 configured to carry out forming for a heating pack 40 in which a heating part 10 is furnished;
a brine provision part 200 configured to supply brine to the heating part 10 of the heating pack 40;
a first adhesion part 300 configured to carry out forming for a first forming sheet 50 by thermally adhering a breathable sheet 51 and a non-breathable sheet 52 to an upper portion and a lower portion of the heating pack 40, respectively;
a second adhesion part 500 configured to carry out forming for a second forming sheet 60 by thermally adhering an outside sheet 61 and an inside sheet 63 to a top surface and a bottom surface of the first forming sheet 50, respectively;
a third adhesion part 600 configured to adhere an earring sheet, which is perforated, to the top surface of the second forming sheet 60 through ultrasonic; and
an outward appearance formation part 700 configured to carry out forming for a mask pack 80 in such a manner as to perforate the second forming sheet 60.

8. The device of claim 7, wherein the pack formation part 100 comprises:
a powder provision member 120 configured to provide a pulp fabric 20 with the heating part by transferring the heating part 10 mixed by a mixer 110;
a thermal adhesion member 140 configured to carry out forming for the heating pack 40 by thermally adhering a non-woven fabric 30 to a top surface of the pulp fabric 20, and sealing the heating part 10 so that the heating part is prevented from being lost;
a punched member 150 configured to punch the heating pack 40 at a fixed interval; and
a cutting member 160 configured to cause the heating pack to be wound on a roller by cutting the heating pack 40 in a large number of heating packs in a fixed width.

9. The device of claim 8, wherein an outer surface of the non-woven fabric 30 facing the pulp fabric 20 is treated with PE coating 31.

10. The device of claim 8, wherein the powder provision member 120 comprises:
a pattern tray 121 which is installed at an upper portion of the pulp fabric 20, and in which input holes 123 are formed in fixed patterns through punching; and
a moving body 131 installed to enable a reciprocating movement at an upper portion of the pattern tray, and configured to cause a large number of the heating parts 10 to be distributed onto a top surface of the pulp fabric 20 via the input holes 123.

11. The device of claim 7, wherein the second adhesion part 500 further comprises a position control sensor part 800,
wherein the position control sensor part 800 rotates so that unwinding of at least any one among the heating pack 40, the breathable sheet 51, the non-breathable sheet 52, and the inside sheet 63, and the earing sheet 70 is regularly maintained by a control part with a position where the outside sheet 61 is unwound as a standard.

12. The device of claim 11, wherein the position control sensor part 800 comprises:
a rotary bracket 810 in which a tension maintenance roller 811 configured to maintain tension of the outside sheet 61 which is unwound is furnished to be rotatable;
a deceleration motor 820 configured to maintain a supply speed of the outside sheet 61 by causing the rotary bracket 810 to rotate and controlling the position where the outside sheet 61 is unwound; and
a pair of sensing sensors 830 configured to sense the outside sheet 61.

13. The device of claim 7, wherein the earing sheet 70 adheres to the second forming sheet so as to unfold in a slanting line form by looking downward with a reference line L arbitrarily formed in the second forming sheet 60 as a standard.
